(19) United States
(12) Patent Application Publication
(10) Pub. No.: US 2009/0107870 A1
(43) Pub. Date: Apr. 30, 2009

(54) Package For Absorbent Articles

(76) Inventors: Jennifer Lyn Tausch, Cincinnati, OH (US); Dean Larry DuVal, Lebanon, OH (US)

Correspondence Address: THE PROCTER & GAMBLE COMPANY;Global Legal Department - IP, Sycamore Building - 4th Floor, 299 East Sixth Street, CINCINNATI, OH, 45202, US

(21) Appl. No.: 12/182,405

(22) Filed: Jul. 30, 2008

Related U.S. Application Data
(60) Provisional application No. 60/983,669, filed on Oct. 30, 2007.

(51) Int. Cl. B65D 73/00; A61F 13/84

(52) U.S. Cl. 206/458; 604/385.02

(57) ABSTRACT

A package for a bedwetting product, the package displaying images of at least one child wearing daytime clothing, the package comprising absorbent articles that are a bedwetting product. The package may optionally comprise images of a child or children that appear to be involved in daytime activities.

### CROSS REFERENCE TO RELATED APPLICATIONS

This application claims the benefit of U.S. Provisional Application No. 60/983,669, filed on Oct. 30, 2007.

### FIELD OF THE INVENTION

The present invention is directed to a package displaying images of at least one child wearing daytime clothing, the package comprising absorbent articles that are a bedwetting product.

### BACKGROUND OF THE INVENTION

About 20% of 5-10 year olds have primary enuresis, the medical name for bedwetting. Bedwetting usually goes away on its own, but until it does, it can be embarrassing and uncomfortable for children. Bedwetting is an issue that millions of families face every night. It is extremely common among children who are under the age of 6, and it can last into the preteen years. Bedwetting can be very stressful for families. Children can feel embarrassed or guilty about wetting the bed and can be anxious about spending the night at a friend's house.

Children who have a bedwetting problem may desire absorbent articles that do not look like traditional diapers, but rather, appear to be more underwear-like. Further, children who have a bedwetting problem need reassurance that they are “normal” and can still fit in with their friends and peers.

Typically, the package that the diapers are packaged in shows designs of infants or toddlers in pajamas, making older children feel that diapers are only for younger children. Thus, there is a need for a package for a bedwetter product that displays images of older children involved in daytime activities, in order to make the wearer feel accepted and included.

### SUMMARY OF THE INVENTION

In accordance with the present invention, a package for a bedwetting product is provided, the package displaying images of at least one child wearing daytime clothing, the package comprising absorbent articles that are a bedwetting product. The package may optionally comprise images of a child or children that appear to be involved in daytime activities, as discussed further hereinafter in the detailed description of the invention.

The present invention is further directed to a method for promoting a feeling of social acceptance for bedwetting children, the method comprising the step of presenting to a person a package comprising at least one absorbent article, the package displaying images of at least one child, wherein the child is wearing daytime clothing, wherein the absorbent article is a bedwetting product.

### BRIEF DESCRIPTION OF THE DRAWINGS

While the specification concludes with claims particularly pointing out and distinctly claiming the subject matter which is regarded as forming the present invention, it is believed that the invention will be better understood from the following description taken in conjunction with the accompanying drawings, in which:

FIG. 1 is package for absorbent articles in accordance with an embodiment of the present invention.

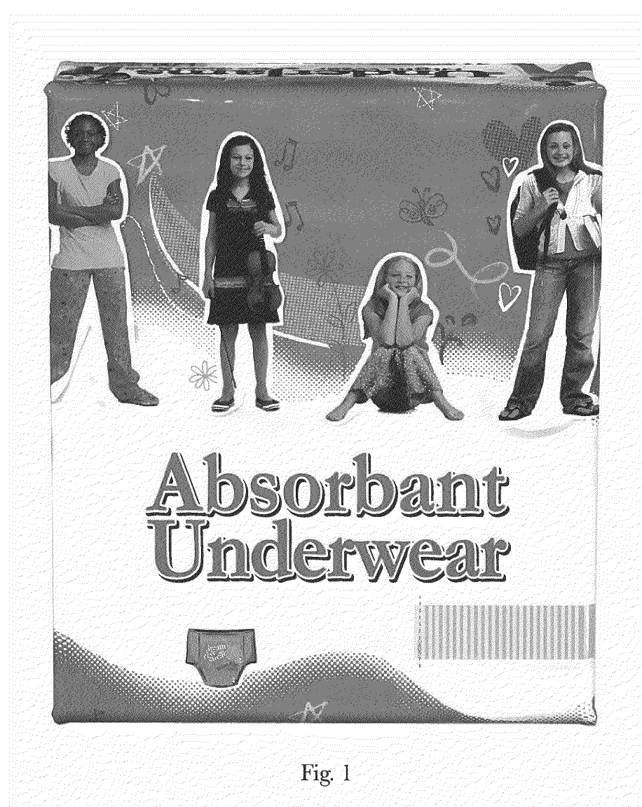

FIG. 2 is package for absorbent articles in accordance with an embodiment of the present invention.

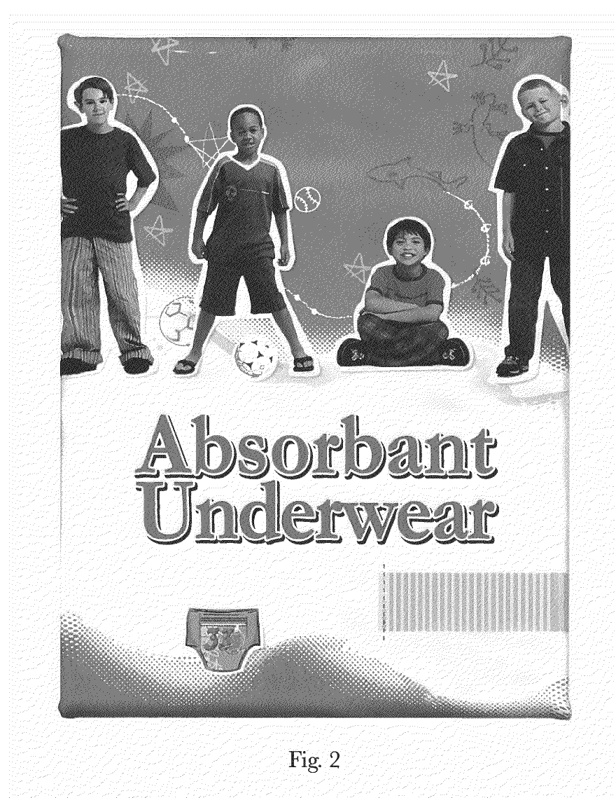

### DETAILED DESCRIPTION OF THE INVENTION

The present invention is directed to a package comprising at least one absorbent article, the package displaying images of at least one child, wherein the child is wearing daytime clothing, wherein the absorbent article is a bedwetting product.

As used herein, the term “absorbent article” refers to devices which are designed to absorb and contain bodily exudates, and, more specifically, refers to devices which are placed within, against, or in proximity to, the body of the wearer to absorb and contain the various exudates discharged from the body.

As used herein the term “diaper” refers to an absorbent article generally used by infants and incontinent persons that is worn about the lower torso of the wearer.

The term “disposable” is used herein to describe absorbent articles which are generally not intended to be laundered or otherwise restored or reused as an absorbent article (in other words, they are generally intended to be discarded after a single use, and, preferably, to be disposed of in an environmentally compatible manner).

As used herein, “image” is an identifying marking, which may include words and/or pictorials and/or designs and/or colors. As used herein, “image” is synonymous with “graphic.”

As used herein, “design” is an identifying feature of the absorbent article, which may include structural or physical elements intended to optimize the product for the targeted age group of the end user. It may also include graphical features of the absorbent article or package such as pictorials, drawings, shapes, bands, patterns, and/or colors intended to simulate underwear products appropriate for the targeted age group of the end user.

A bedwetting child who uses a disposable absorbent article is typically one who is toilet trained during the day, but still has accidents (urine only) at night, and is older than 4 or 5 years old. The child's incontinence may be a result of chemical imbalance, physiological development (e.g. delayed bladder development), psychological, or sociological issues. This incontinence issue presents a social restriction on the children who suffer from it, precluding the ability to participate in sleepovers or overnight camps without fear of overnight accidents.

Feelings of embarrassment and the need for discretion are very important for older children who have a bedwetting problem. Caregivers of bedwetting children are constantly concerned about their child's bedwetting issues and frequently discuss such problems with pediatricians in order to understand if their child is developing properly and getting enough sleep. Caregivers are also seeking solutions to stop the bedwetting problem and solutions to cope with the emotional side effects their child suffers.

A package for a bedwetting product that displays images of a child or children wearing daytime clothing conveys to a bedwetting child that the product is inconspicuous.

Further, the package may display images of two or more children. Inclusion of multiple children demonstrates acceptance and inclusion by other kids to the bedwetting child.

Displaying images of older children on the package also communicates feelings of acceptance and optimism to the bedwetting child. The older children displayed on the package may be children who are four years or older, five years or older, six years or older. The children displayed on the package may be children who are 12 years or younger, 11 years or younger, 10 years or younger.

Displaying images of children involved in daytime activities indicates that bedwetting is a normal problem, and bedwetters are able to be accomplished children. The fun and relaxed images of children make the bedwetter feel that the product will allow him/her to live his/her life, and the condition is a normal problem that many children face. Further, the inclusion of multiple and diverse children on the package communicates that the children can be themselves, which helps increase their optimism about the condition. The daytime activities that the children are engaged in include, but are not limited to, playing an instrument, playing a sport, carrying a backpack, reading a book, dancing, holding a skateboard, drawing, painting, sketching, studying, thinking, swimming, snorkeling, flying a kite, shopping, running, smiling, exhibiting confidence in themselves, etc.

The package may further comprise images of children in nighttime clothing, such as pajamas. These images indicate to the bedwetting children that they can feel comfortable going to sleepovers with their friends and not feel worried or embarrassed.

A package comprising a bedwetting absorbent article that displays images of children are wearing daytime clothing, involved in daytime activities communicates normalcy of the bedwetting condition and tells the bedwetting child that they are not alone in having this problem. Further, caregivers can use the package as a discussion aid with children to reassure him/her that they are not alone and children of all ages with all different interests wet the bed.

The package may have other design features that indicate to the caregiver and bedwetting child that this product is not like diapers for younger kids. Most diaper packages have a glossy appearance over the entire package. The package of the present invention may have at least a portion of the package displaying a matte finish to differentiate this product from traditional diaper products. For instance, in one embodiment, the package may display the images of the children in a gloss finish, and the rest of the package may be in a matte finish. Alternatively, the images of the children and the words on the package may be in a gloss finish and the rest of the package may be in a matte finish.

The absorbent article contained in the package may also have design features for older children who are having a bedwetting problem and want disposable absorbent articles to look more like underwear than diapers. The absorbent article may be substantially or entirely colored, thus appearing to look more like underwear. Also, the absorbent articles may have gender specific graphics. Further, the absorbent articles contained within the package may comprise at least two different designs, which is similar to how actual underwear is packaged. The designs of the absorbent article may be similar to or the same as the designs of actual underwear. Optionally, the different designs may be complementary to each other, also similar to actual underwear. One way that the designs may be complementary to each other is to have at least one color the same between the two designs. Alternatively, the waist bands of the two designs may match or contrast to provide the effect of actual underwear.

The absorbent article may also display images that are linked to the daytime activities displayed on the package. For instance, a package that displays images of children drawing or sketching may contain absorbent articles showing line drawings, doodling, and sketches. Further, a package that displays images of a child playing a sport may contain absorbent articles showing the ball or other accessory used in the sport.

The absorbent article may comprise a chassis providing the comfort and look of underpants and the protection of a diaper, especially designed for older children with a bedwetting problem. For instance, the absorbent article may be designed to have a narrow crotch width and/or a thin, flexible core and/or stretch across most of the waist portion and/or gender specific graphics and/or all over color.

A bulky, large disposable absorbent article might relieve containment concerns, but negate discretion, one of the key benefits of using an overnight product. Ideally, no one would know that the child was wearing a disposable absorbent article instead of underwear, providing the child with an underwear-like experience without the mess. Different product attributes that would help achieve discretion are the thickness of the product, the sound the product makes (diaper-like products are audible), age-appropriate graphics, characters, and colors, and shorter pitch. While it needs to achieve these means of discretion, it still needs to perform well in overnight containment situations, since a single accident can cause significant embarrassment for the child.

In order to further reassure children that bedwetting is a normal problem that many children deal with and increase his/her self-esteem, the package of the present invention may be sold together with actual underwear. The bedwetter package may be placed in the underwear aisle (or, alternatively, the underwear package may be placed in the same aisle that the bedwetter product is normally placed in), and the bedwetter package and/or absorbent article design may be the same as or similar to the underwear package and/or underwear design. Thus, the daytime experience and nighttime experience has a common theme and feeling of normalcy for the bedwetter.

Further, the package of the present invention may be marketed or co-marketed together with actual underwear. For instance, there may be coupons or promotional material found in the underwear aisle and/or the absorbent article aisle and/or any other store aisle advertising the bedwetter package or the corresponding underwear package. The coupons or promotional material may contain the same designs, characters, patterns, and/or colors as the bedwetter package or the underwear package. The coupons or promotional materials may communicate normalcy of the bedwetting problem and provide caregivers with an opportunity to reward children for day-time progress while maintaining a level of nighttime protection.

The underwear and/or the bedwetter product may further provide educational or informational literature providing facts on bedwetting to reassure the child and/or caregiver that bedwetting is a normal condition, in order to increase the child's self-esteem.

The present invention is also directed to a method for promoting a feeling of social acceptance for bedwetting children. Presenting a bedwetter and the caregiver a package for a bedwetting product that displays images of children wearing daytime clothing promotes a feeling of acceptance and inclusion for the bedwetting child. Presenting a package displaying images of children involved in daytime activities promotes a feeling of optimism and normalcy to the bedwetting child.

All patents and patent applications (including any patents which issue thereon) assigned to the Procter & Gamble Company referred to herein are hereby incorporated by reference to the extent that it is consistent herewith.

The dimensions and values disclosed herein are not to be understood as being strictly limited to the exact numerical values recited. Instead, unless otherwise specified, each such dimension is intended to mean both the recited value and a functionally equivalent range surrounding that value. For example, a dimension disclosed as “40 mm” is intended to mean “about 40 mm.”

All documents cited in the Detailed Description of the Invention are, in relevant part, incorporated herein by reference; the citation of any document is not to be construed as an admission that it is prior art with respect to the present invention. To the extent that any meaning or definition of a term in this document conflicts with any meaning or definition of the same term in a document incorporated by reference, the meaning or definition assigned to that term in this document shall govern.

While particular embodiments of the present invention have been illustrated and described, it would be obvious to those skilled in the art that various other changes and modifications can be made without departing from the spirit and scope of the invention. It is therefore intended to cover in the appended claims all such changes and modifications that are within the scope of this invention.

What is claimed is:

1. A package comprising at least one absorbent article, said package displaying an image of a child, wherein said child is wearing daytime clothing, wherein said absorbent article is a bedwetting product.

2. The package of claim 1, wherein said child appears to be between the ages of from about 4 to about 12 years old.

3. The package of claim 1, wherein said child appears to be involved in a daytime activity.

4. The package of claim 3, wherein said daytime activity is selected from the group consisting of playing an instrument, playing a sport, carrying a backpack, reading a book, dancing, holding a skateboard, drawing, painting, sketching, studying, thinking, swimming, snorkeling, flying a kite, shopping, and running.

5. The package of claim 1, displaying an image of at least two children.

6. The package of claim 1, further displaying an image of at least one child wearing nighttime clothing.

7. The package of claim 1, wherein said image of at least one child is displayed in a gloss finish.

8. The package of claim 7, wherein at least a portion of the background is a matte finish.

9. The package of claim 1 comprising at least two absorbent articles, wherein said at least two absorbent articles display different designs.

10. The package of claim 9, wherein said different designs comprise at least one color the same.

11. The package of claim 3, wherein said absorbent article displays designs that are linked to said daytime activity displayed on said package.

12. A method for promoting a feeling of social acceptance for bedwetting children, said method comprising the step of presenting to a person a package comprising at least one absorbent article, said package displaying an image of a child, wherein said child is wearing daytime clothing, wherein said absorbent article is a bedwetting product.

13. The method of claim 11, wherein said child appears to be involved in a daytime activity.

14. A method for promoting a feeling of social acceptance for bedwetting children, said method comprising the steps of:
a. presenting to a person an absorbent article package comprising at least one absorbent article, said package displaying an image of a child, wherein said child is wearing daytime clothing; and
b. presenting to a person an underwear package comprising at least one pair of underwear, said package displaying an image of a child, wherein said child is wearing daytime clothing;
wherein said absorbent article package and/or said absorbent article and said underwear package and/or said underwear comprise similar images.

15. The method of claim 14, wherein said absorbent article is a bedwetting product.

16. The method of claim 14, wherein said absorbent article package and said underwear package are displayed in the same aisle.

17. The method of claim 14, wherein said image of a child on said absorbent article package and said underwear package appears to be involved in a daytime activity.
